(19) United States
(12) Patent Application Publication
(10) Pub. No.: US 2020/0101219 A1
(43) Pub. Date: Apr. 2, 2020

(54) Injection Device

(71) Applicant: Jabil Circuit (Shanghai) Ltd., Shanghai (CN)

(72) Inventors: Zhe-Heng Wang, Shanghai (CN); Ying Li, Shanghai (CN)

(73) Assignee: Jabil Circuit (Shanghai) Ltd., Shanghai (CN)

(21) Appl. No.: 16/558,844

(22) Filed: Sep. 3, 2019

(30) Foreign Application Priority Data
Sep. 27, 2018 (CN) 201811131743.7

(51) Int. Cl. A61M 5/142; A61M 5/168; A61M 5/172

(52) U.S. Cl. CPC A61M 5/14248; A61M 5/16831; A61M 2205/8206; A61M 2005/14252; A61M 2205/3334; A61M 5/1723

(57) ABSTRACT

An injection device adapted for adhering to skin of a user to inject a liquid into the user includes an adhesive patch, and an injection module. The adhesive patch has a patch body and a hollow region that is formed in the patch body. The patch body has opposite first and second surfaces, the second surface being adapted for adhering to the skin of the user. The injection module includes a connecting seat and a catheter connected to the connecting seat and having a secured end portion and an injection end portion that is opposite to the secured end portion. The catheter extends, at an angle to the adhesive patch, from the connecting seat through the hollow region to a side of the adhesive patch corresponding to the second surface.

### CROSS-REFERENCE TO RELATED APPLICATION

This application claims priority of Chinese Invention Patent Application No. 201811131743.7, filed on Sep. 27, 2018.

### FIELD

The disclosure relates to an injection device, more particularly to an injection device for intravenous injections.

### BACKGROUND

A catheter is a medication injection equipment in modern medical treatment. The catheter remains inserted in a patient's vein to provide blood, medication, nutrition solution, or other liquid substances for long periods of time, which reduces discomfort from repeated injections or the fear that the patient may have towards the injections. As shown in FIG. 1, a conventional intravenous injection device 1 includes a catheter 11, an adhesive film 12 connected to the catheter 11, and a release liner 13 disposed on the adhesive film 12. The process of performing intravenous injections includes the following steps. First, a medical staff holds the catheter 11 and pierces the skin with the catheter 11 at an angle of 30 to 40 degrees, and then inserts the catheter 11 into a vein at 15 degrees. Then, with one hand securing the catheter 11, the medical staff removes the release liner 13 using the other hand. Finally, the adhesive film 12 is adhered to the skin surrounding the catheter 11 so as to secure the catheter 11. However, when the medical staff is holding the catheter 11, the adhesive film 12 adhered to the catheter 11 will also be touched, which may cause the adhesive film 12 to become creased and may decrease the adhesiveness of the adhesive film 12.

### SUMMARY

Therefore, the object of the disclosure is to provide an injection device that can alleviate at least one of the drawbacks of the prior art.

According to a first aspect of the disclosure, an injection device adapted for adhering to skin of a user to inject a liquid into the user includes an adhesive patch and an injection module.

The adhesive patch has a patch body and a hollow region that is formed in the patch body. The patch body has opposite first and second surfaces, the second surface being adapted for adhering to the skin of the user.

The injection module includes a connecting seat that is disposed on the first surface, and a catheter that is connected to the connecting seat. The catheter has a secured end portion that is disposed in the connecting seat, and an injection end portion that is opposite to the secured end portion. The catheter extends, at an angle to the adhesive patch, from the connecting seat through the hollow region to a side of the adhesive patch corresponding to the second surface.

According to a second aspect of the disclosure, an injection device adapted for adhering to the skin of a user to inject a liquid into the user includes an adhesive patch, a catheter, and a release member.

The adhesive patch has a patch body and a hollow region that is formed in the patch body. The patch body has opposite first and second surfaces.

The catheter extends at an angle to the adhesive patch from the first surface, through the hollow region, to a side of the adhesive patch corresponding to the second surface.

The release member removably covers the second surface of the patch body.

According to a third aspect of the disclosure, an injection device adapted for adhering to the skin of a user to inject a liquid into the user includes an adhesive patch and a catheter.

The adhesive patch has a patch body and a hollow region that is formed in the patch body. The patch body has opposite first and second surfaces.

The catheter has a secured end portion that is disposed at the first surface of the patch body and an injection end portion that is disposed at the second surface of the patch body and for piercing the skin of the user.

### BRIEF DESCRIPTION OF THE DRAWINGS

Other features and advantages of the disclosure will become apparent in the following detailed description of the embodiments with reference to the accompanying drawings, of which:

FIG. 1 is a schematic view of conventional intravenous injection device;

FIG. 2 is a schematic view of a first embodiment of an injection device according to the disclosure;

FIG. 3 is a perspective partially exploded view of the first embodiment illustrating an outer casing detached;

FIG. 4 is a side view of FIG. 3, illustrating a catheter of the first embodiment extending at an angle through a hollow region of an adhesive patch of the first embodiment;

FIG. 5 is an exploded perspective view of the first embodiment;

FIG. 6 is a top view of the first embodiment with the outer casing removed;

FIG. 7 is a top view, illustrating folding portions of a circuit board of the first embodiment unfolded;

FIG. 8 is a top view, illustrating the folding portions of the circuit board of the first embodiment folded;

FIG. 9 is a fragmentary side view, illustrating an electrically insulating layer of the first embodiment disposed between the folding portion and a main board portion of the circuit board;

FIG. 10 is bottom view, illustrating the release member of the first embodiment not yet removed from the adhesive patch;

FIG. 11 is a bottom view, illustrating the first embodiment with the release member removed and an actuating module and a photoplethysmography module of the first embodiment exposed; and

FIG. 12 is a perspective view of a second embodiment of the injection device according to the disclosure.

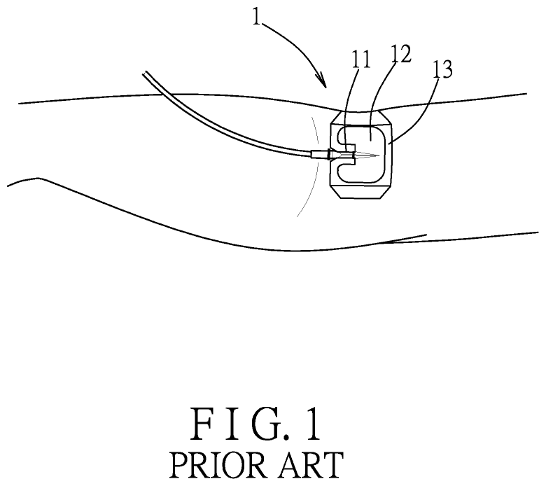

### DETAILED DESCRIPTION

Before the present invention is described in greater detail, it should be noted that where considered appropriate, reference numerals or terminal portions of reference numerals have been repeated among the figures to indicate corresponding or analogous elements, which may optionally have similar characteristics.

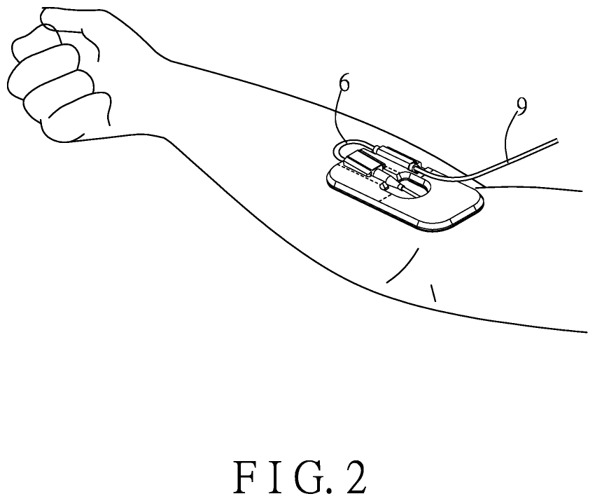

Referring to FIGS. 2 to 5, a first embodiment of an injection device according to the disclosure is configured to adhere to skin of a user (e.g., a patient) and to connect to an infusion tube 9 to inject a liquid into a vein of the user. The injection device includes an adhesive patch 2, an outer casing 3, an injection module 4, a monitoring unit 5, a connecting tube 6, a release member 7 and a sponge unit 8. In this embodiment, the injection device is exemplified to be configured to adhere to an upper limb of the user to perform injection, but is not limited thus and can also be adhered to lower limbs, or other regions of a human body suitable for intravenous injection.

The adhesive patch 2 has a patch body 21 and a hollow region 22 that is formed in a central area of the patch body 21. The patch body 21 has opposite first and second surfaces 211, 212, opposite first and second edges 213, 214 being shorter edges of the patch body 21, a first load region 215 defined on the first surface 211 between the first edge 213 and the hollow region 22, and a second load region 216 defined on the first surface 211 between the second edge 214 and the hollow region 22. In this embodiment, the patch body 21 is substantially shaped as a rounded rectangle to avoid causing the user discomfort from sharp corners poking into the skin. The first and second surfaces 211, 212 are adhesive, and the second surface 212 is configured to adhere to the skin of the user. The hollow region 22 has a size that is suitable for a finger of a medical staff to extend through.

The outer casing 3 covers the first surface 211 of the adhesive patch 2, and defines a receiving space 31 that receives a portion of the injection module 4 and the monitoring unit 5. The outer casing 3 has two hollow portions 32 disposed on a side of the outer casing 3. The hollow portions 32 allow the connecting tube 6 to extend therethrough, such that the connecting tube 6 is exposed from the outer casing 3 to allow the medical staff to easily monitor liquid inside.

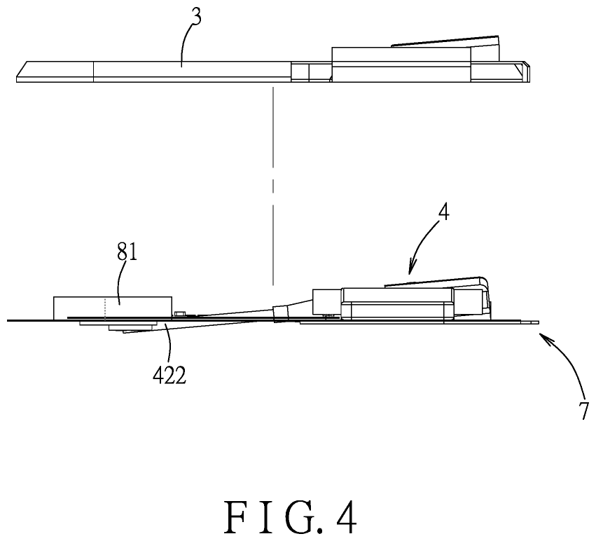

Referring to FIGS. 4 to 6, the injection module 4 includes a connecting seat 41 that is disposed on the first surface 211 and in the first load region 215 of the patch body 21, and a catheter 42 that is connected to the connecting seat 41. The catheter 42 has a secured end portion 421 that is disposed in the connecting seat 41, and an injection end portion 422 that is opposite to the secured end portion 421. The catheter 42 extends, at an angle to the adhesive patch 2, from the connecting seat 41 through the hollow region 22 to a side of the adhesive patch 2 corresponding to the second surface 212. The medical staff can hold the catheter 42 through the hollow region 22, and pierce the skin of the user with the injection end portion 422 to perform an injection procedure. This allows the injection procedure to be performed without the patch body 21 being touched and thus creased by the medical staff.

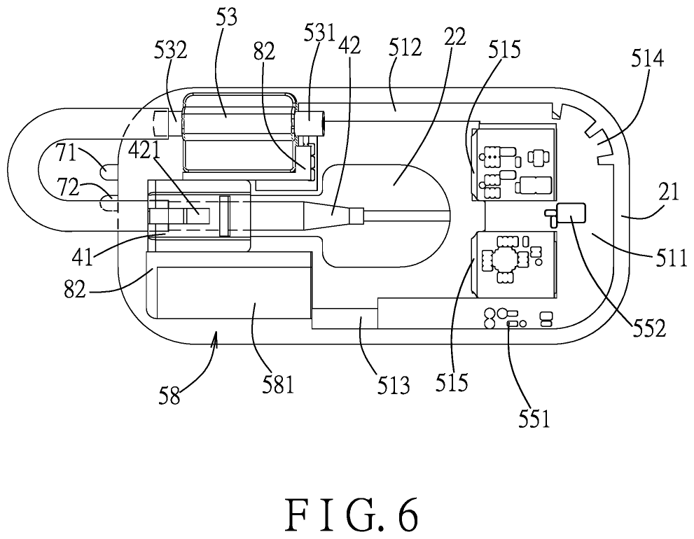

Referring to FIGS. 6, 7, 8, and 11, the monitoring unit 5 includes a circuit board 51, a processing module 52, a flow meter 53, a photoplethysmography module 54, a temperature sensing module 55, a three-axis sensor 56, a signal light module 57, a power supply module 58, and an actuating module 59.

In this embodiment, the circuit board 51 is a flexible printed circuit (FPC) board configured to allow electronic components to be installed thereon, and has a main board portion 511, a first extending portion 512, a second extending portion 513, a plurality of slits 514, and a folded portion 515. In this embodiment, the circuit board 51 includes two folded portions 515. The main board portion 511 and the folded portions 515 are all configured to allow electronic components to be installed thereon. The slits 514 are formed on a periphery of the main board portion 511. In this embodiment, the periphery of the main board portion 511 is aligned with a part of a periphery of the patch body 21.

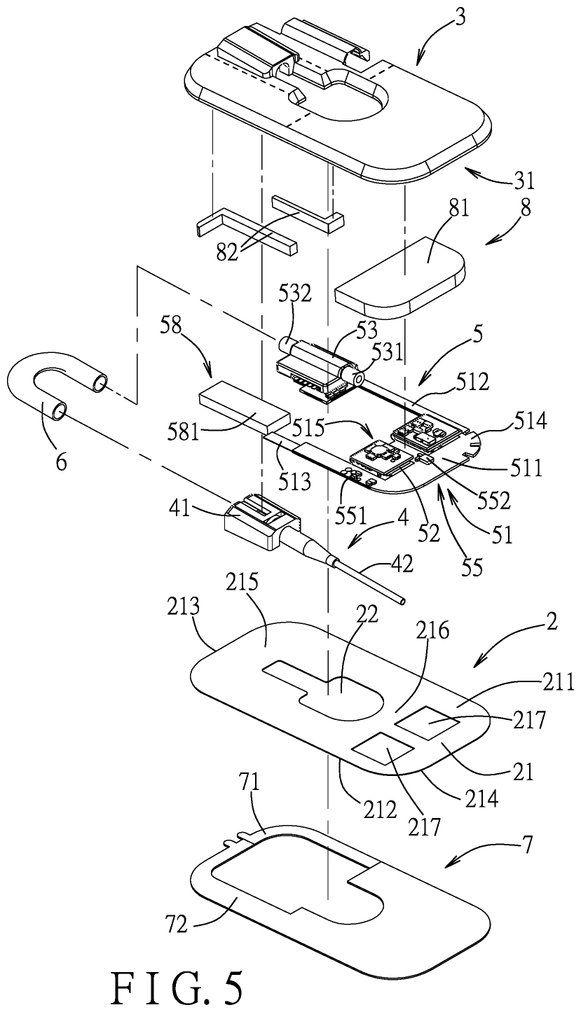

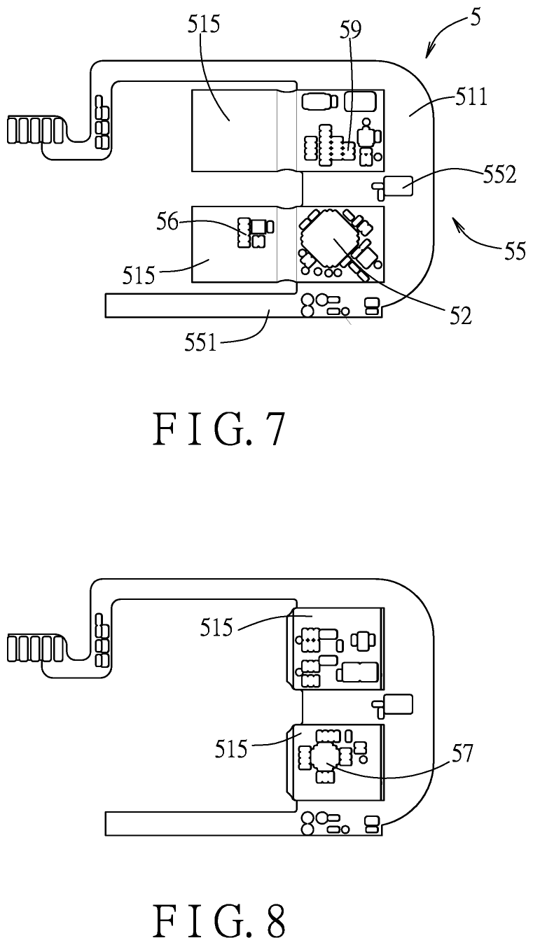

Referring back to FIG. 5, in this embodiment, the main board portion 511 has a first side formed with the slits 514, and a second side opposite to the first side. The slits 514 improve the flexibility of the circuit board 51 and allow the injection device to adhere to the skin of the user in a more comfortable way. Each of the first and second extending portions 512, 513 is connected to and extends from the second side of the main board portion 511 in a direction away from the main board portion 511, is strip-shaped, and has an end section that is opposite to the main board portion 511 and that is disposed in the first load region 215 of the patch body 21. A plurality of electronic components is disposed at the main board portion 511 and the end sections of the first and second extending portions 512, 513, and not disposed between the main board portion 511 and the end section of each of the first and second extending portions 512, 513. Further referring to FIGS. 7 and 11, specifically, the flow meter 53 is disposed on the end section of the first extending portion 512, the power supply module 58 is disposed on the end section of the second extending portion 513, and the processing module 52, the photoplethysmography module 54, the temperature sensing module 55 and the actuating module 59 are disposed on the main board portion 511. A section of each of the first and second extending portions 512, 513 between the main board portion 511 and the respective end section is not installed with any components thereon, and thus has more flexibility, hence allowing a portion of the patch body 21 corresponding in position to these sections to retain more flexibility also.

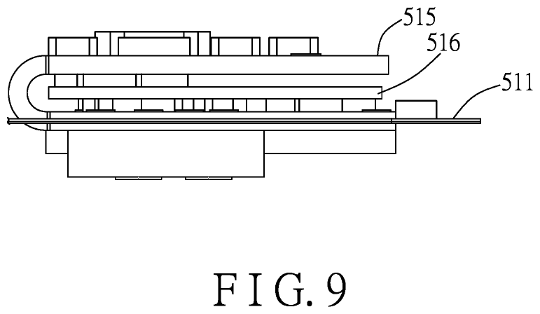

Referring to FIGS. 7 to 9, the folded portions 515 are connected to and disposed over the main board portion 511, and are for electrically connecting the three-axis sensor 56, the signal light module 57, and other electrical components. In this embodiment, the folded portions 515 extend from the first side of the main board portion 511 and folds over the top of the main board portion 511, reducing the planar area of the circuit board 51. An electrically insulating layer 516 is disposed between the folded portions 515 and the main board portion 511 to prevent electrical components disposed thereon to touch and short-circuit. The electrically insulating layer 516 may be made of a material such as mylar. It should be noted that FIG. 7 is a schematic view for illustrating connection between the folded portions 515 and the main board portion 511 and exemplary positions respectively of the processing module 52, the three-axis sensor 56 and the actuating module 59 on the circuit board 51, and a real structure of the circuit board 51 is shown in FIG. 8.

The processing module 52 is disposed on the main board portion 511 of the circuit board 51, includes a conventional wireless communication element that uses, for example, WiFi or Bluetooth technology, and is configured to wirelessly communicate with an external monitoring device (not shown) to wirelessly transmit data obtained by the monitoring unit 5 to the external monitoring device for the medical staff to keep track of. The external monitoring device may be a smart phone, a tablet computer, a wearable device, a desktop computer, a notebook computer, etc., but is not limited thus.

Referring to FIGS. 2 and 6, the flow meter 53 is electrically connected to the processing module 52 through the circuit board 51. The flow meter 53 has an input port 531 facing the main board portion 511, and an output port 532 opposite to the input port 531. The input port 531 is configured to be connected to the infusion tube 9, and the output port 532 is connected to the connecting tube 6 which is also connected to the secured end portion 421 of the catheter 42. The liquid to be injected into the user first enters the flow meter 53 through the infusion tube 9, and the flow meter 53 can measure a volumetric flow rate and a flow speed of the liquid flowing into the user. The result of the measurement is transmitted to the external monitoring device through the processing module 52 to allow the medical staff to monitor in real time the volumetric flow rate and the flow speed of the liquid. Then, the liquid is injected into the vein of the user through the connecting tube 6 and the catheter 42.

Referring to FIG. 11, the photoplethysmography module 54 is disposed on a bottom surface of the main board portion 511 of the circuit board 51 (i.e., a surface facing the skin of the user when the injection device adheres to the user), electrically connected to the processing module 52 through the circuit board 51, exposed from the second surface 212 of the patch body 21 through a corresponding one of two square openings 217 in the patch body 21, and proximate to the injection end portion 422 of the catheter 42. The photoplethysmography module 54 includes a light-proof cover 541 having first and second light-transmissible windows 541a, 541b, an transmitter 542 corresponding in position to the first light-transmissible window 541a, and a receiver 543 corresponding in position to the second transparent window 541b. The first and second light-transmissible windows 541a, 541b may be made of a transparent material or be hollow. The transmitter 542 transmits a sensing light beam to the skin and vein of the user, and the receiver 543 receives the sensing light beam reflected by the skin and vein of the user. Since the heart beat causes a periodic change in the blood flow rate per unit area in the vein, and hence an absorbed amount of the sensing light absorbed by hemoglobin in the blood also changes periodically, the photoplethysmography module 54 can calculate the absorbed amount of the sensing light that has been absorbed and transmit a photoplethysmography signal that is related to the absorbed amount of the sensing light to the external monitoring device through the processing module 52 so that the medical staff may monitor, in real time, blood oxygen concentration of the user according to the absorbed amount of the sensing light. The photoplethysmography module 54 can also be used to detect if the skin is having an allergic reaction, and if the injection end portion 422 of the catheter 42 is leaking.

Referring to FIG. 6, the temperature sensing module 55 includes a first temperature sensor 551 and a second temperature sensor 552. The first temperature sensor 551 is disposed on the main board portion 511, proximate to the second extending portion 513, and is used for sensing a first temperature of a position of the skin apart from the injection end portion 422 of the catheter 42. The second temperature sensor 552 is disposed on the main board portion 551 and between the folding portions 515, and is used for sensing a second temperature of a position of the skin proximate to the injection end portion 422 of the catheter 42 or to an injection position where the injection end portion 422 penetrates the skin. Since there is a positional difference between the first and second temperature sensors 551, 552, a difference between the first and second temperatures can be used to determine an injection status of the user. For example, if the second temperature is significantly higher than the first temperature, this could indicate that the skin around the injection position is inflamed. The temperature information measured by the temperature sensing module 52 is transmitted to the external monitoring device through the processing module 52 for use of the medical staff.

Referring to FIG. 7, the three-axis sensor 56 is disposed on a first surface of one of the folding portions 515 that faces the main board portion 511, and is used to detect a tilting angle of the injection device. The tilting angle is transmitted to the external monitoring device through the processing module 52 for the medical staff to monitor and prevent excessive tilt of the injection device causing blood of the user to reflux into the catheter 42 or leak from the injection position.

Referring to FIG. 8, the signal light module 57 is disposed on a second surface of one of the folding portions 515 that is opposite to the first surface and proximate to the outer casing 3. In this embodiment, the signal light module 57 is a circuit switch with a light-emitting diode. When the injection device is adhered to the skin of the user, the signal light module 57 is for the user to press to turn on or off the injection device, with the light-emitting diode emitting light to indicate that the injection device is in an on state.

Referring to FIGS. 5 and 11, the power supply module 58 includes a rechargeable battery 581 that is disposed on the end section of the second extending portion 513, and a charging terminal 582 that is disposed on and exposed from the light-proof cover 541 of the photoplethysmography module 54 and the patch body 21, and that is electrically connected to the rechargeable battery 581 through the circuit board 51. The rechargeable battery 581 is configured to supply, though the circuit board 51, electricity to electronic components of the monitoring unit 5 including the processing module 52, the flow meter 53, the photoplethysmography module 54, the temperature sensing module 55, the three-axis sensor 56, the signal light module 57, and the actuating module 59. As the light-proof cover 541 is made of a material having a greater stiffness than the circuit board 51 and the adhesive patch 2, having the charging terminal 582 disposed on the light-proof cover 541 helps with connecting an external charger (not shown) to the charging terminal 582. The external charger may be, for example, a charging stand having a mechanism that can securely hold the injection device.

The actuating module 59 is disposed on the main board portion 511 of the circuit board 51 and under the processing module 52. The actuating module 59 is exposed from the second surface 212 of the patch body 21 through a corresponding one of the square openings 217 of the patch body 21. The actuating module 59 is configured to detect a light and to actuate at least one electronic component on the circuit board 51 according to the detection. In this embodiment, the actuating module 59 is a light detecting device used for detecting a light to actuate the processing module 52, the flow meter 53, the photoplethysmography module 54, the temperature sensing module 55, the three-axis sensor 56, and the signal light module 57. The actuating module 59 is kept in electrical connection with the power supply module 58 before use of the injection device in order to receive power from the power supply module 58 to continuously detect light.

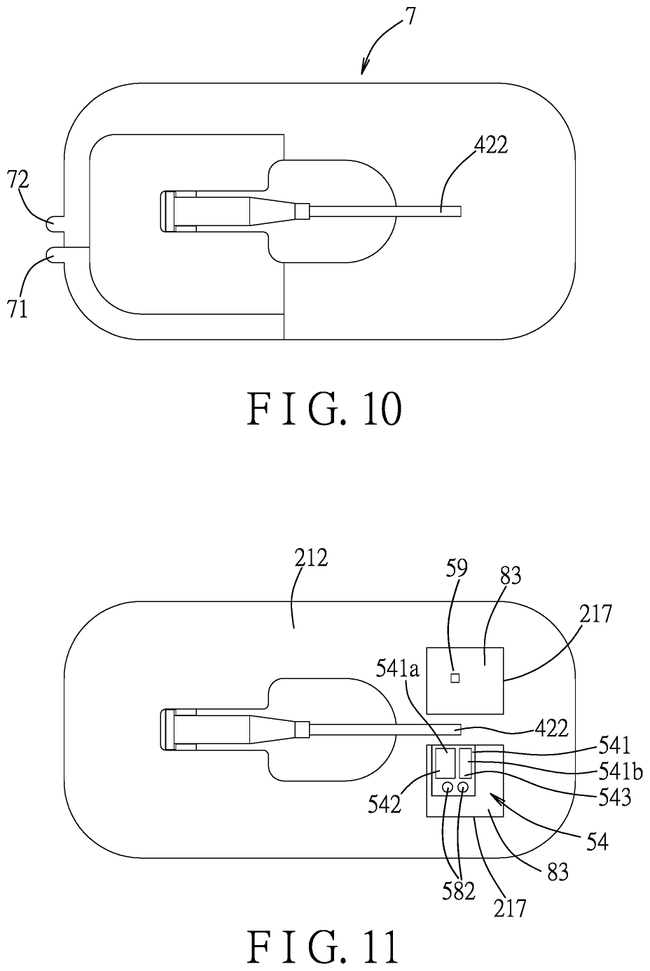

Referring to FIGS. 5 and 10, the release member 7 removably covers the second surface 212 of the patch body 21 to shield the actuating module 59. In this embodiment, the release member 7 is a release liner. The release member 7 includes a first release portion 71 and a second release portion 72 that may be removed separately to allow the position of the injection device on the skin of the user to be more easily adjusted when adhering the adhesive patch 2 to the skin. The first release portion 71 has a smaller area than the second release portion 72, is disposed proximate to the edge of the first load region 215, has a long, thin strip-shape and is disposed correspondingly under the flow meter 53. The second release portion 72 has a larger area, and has a long, thin section disposed in the first load region 215 and a larger section disposed correspondingly under the actuating module 59 and the photoplethysmography module 54 in the second load section 216. When using the injection device, the user may remove the first release portion 71 first to adhere to the skin of the user a section of the adhesive patch 2 corresponding to the flow meter 53. Since the flow meter 53 is a stiffer component, the section of the adhesive patch 2 corresponding to the flow meter 53 can be more securely adhered to the skin of the user. A section of the adhesive patch 2 still covered by the second release portion 72 can still be adjusted to a certain degree by the medical staff before the second release portion 72 is also removed and the injection device is fully adhered to the skin of the user. Once the second release portion 72 is removed, the actuating module 59 which would have been covered by the second release portion 72 can detect a change in ambient light, and automatically actuates the processing module 52, the flow meter 53, the photoplethysmography module 54, the temperature sensing module 55, the three-axis sensor 56, and the signal light module 57 without needing manual input from the user. In other embodiments, the actuating module 59 may only actuate the processing module 52, with the processing module 52 subsequently actuating the other electrical components. To switch off the injection device, the user only need to press on a position of the outer casing 3 corresponding in position above the light signal module 57 so that the outer casing 3 presses on the light signal module 57. Specifically, at least a region of the outer casing 3 corresponding in position to the signal light module 57 may be light-transmissible or formed with holes so that the user may see light emitted by the light-emitting diode of the signal light module 57.

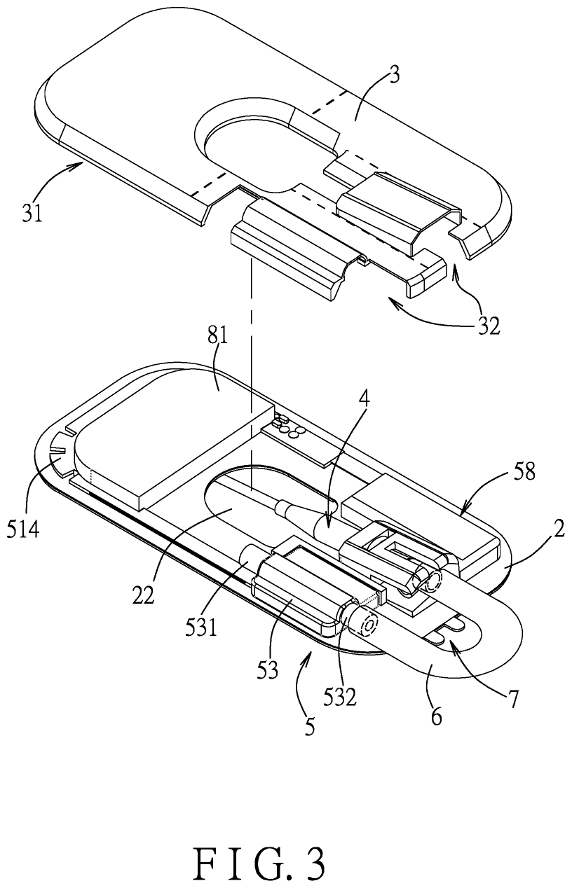

Referring to FIGS. 3, 5, and 11, the sponge unit 8 is used to provide cushioning in the injection device, and includes a first sponge 81 covering the folded portions 515, two second sponges 82 disposed respective between the battery 581 and the connecting seat 41 and between the connecting seat 41 and the flow meter 53, and two third sponges 83 respectively covering the actuating module 59 and the photoplethysmography module 54. Each of the actuating module 59 and the photoplethysmography module 54 are partially exposed from a respective one of the third sponges 83.

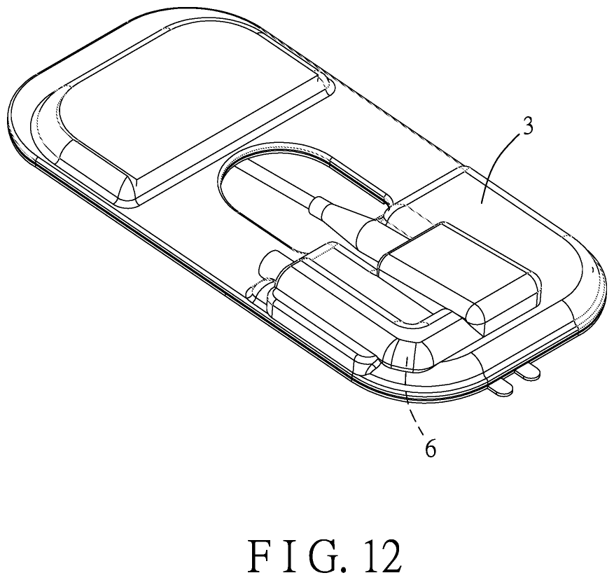

Referring to FIG. 12, a second embodiment of the injection device according to the disclosure is substantially the same as the first embodiment, the difference being that the outer casing 3 of the second embodiment does not have hollow portions 32 and that the connecting tube 6 is fully received within the outer shell 3.

In sum, compared with the conventional injection device 1, which has an adhesive film 12 that can be easily creased during the process of performing intravenous injection, the injection device of this disclosure has the patch body 21 formed with the hollow region 22, allowing the medical staff to avoid touching the patch body 21 while performing the injection, which enables the entirety of the patch body 21 to remain adhered to the skin. Moreover, the monitoring unit 5 can detect the volumetric flow rate and flow speed of the liquid, the blood oxygen concentration, whether the catheter 42 is leaking, whether the skin is having an allergic reaction, etc., which helps the medical staff obtain physiological information of the user.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. An injection device adapted for adhering to skin of a user to inject a liquid into the user, said injection device comprising:
an adhesive patch having a patch body and a hollow region that is formed in said patch body, said patch body having opposite first and second surfaces, said second surface being adapted for adhering to the skin of the user; and
an injection module including a connecting seat that is disposed on said first surface, and a catheter that is connected to said connecting seat, said catheter having a secured end portion that is disposed in said connecting seat, and an injection end portion that is opposite to said secured end portion, said catheter extending, at an angle to said adhesive patch, from said connecting seat through said hollow region to a side of said adhesive patch corresponding to said second surface.

2. The injection device as claimed in claim 1, further comprising a monitoring unit including a circuit board that is disposed on said first surface of said patch body, wherein said circuit board is a flexible printed circuit board, and has a main board portion that is formed with a plurality of slits on a periphery of said main board portion.

3. The injection device as claimed in claim 2, wherein said periphery of said main board portion is aligned with a part of a periphery of said patch body.

4. The injection device as claimed in claim 2,
wherein said main board portion has a first side formed with said slits, and a second side opposite to said first side,
wherein said circuit board further has an extending portion that is connected to and extends from said second side of said main board portion in a direction away from said main board portion, that is strip-shaped, and that has an end section opposite to said main board portion, and
wherein a section of said extending portion between said main board portion and said end section is not installed with any components thereon.

5. The injection device as claimed in claim 4, wherein a plurality of electronic components are disposed at said main board portion and said end section of said extending portion.

6. The injection device as claimed in claim 2, wherein said monitoring unit further includes a photoplethysmography module, said photoplethysmography module including a light-proof cover having first and second light-transmissible windows, an transmitter corresponding in position to said first light-transmissible window, and a receiver corresponding in position to said second transparent window.

7. The injection device as claimed in claim 6, wherein said power supply module includes a rechargeable battery configured to supply electricity to electronic components of said monitoring unit, and a charging terminal exposed from said patch body.

8. The injection device as claimed in claim 6, wherein said power supply module includes a rechargeable battery configured to supply electricity to electronic components of said monitoring unit, and a charging terminal disposed on and exposed from said light-proof cover of said photoplethysmography module and electrically connected to said rechargeable battery through said circuit board.

9. The injection device as claimed in claim 1, further comprising a flexible printed circuit board having a main board portion, and an extending portion that extends from said main board portion in a direction away from said main board portion, that is strip-shaped, and that has an end section opposite to said main board portion, wherein a section of said extending portion between said main board portion and said end section is not installed with any components thereon.

10. The injection device as claimed in claim 9, further comprising a plurality of electronic components disposed at said main board portion and said end section of said extending portion.

11. The injection device as claimed in claim 1, further comprising:
a circuit board configured to allow at least one electronic component to be installed thereon; and
an actuating module disposed on said circuit board and exposed from said second surface of said patch body, said actuating module being configured to detect a light and to actuate the at least one electronic component on said circuit board according to the detection.

12. The injection device as claimed in claim 11, further comprising a release member removably covering said second surface of said patch body to shield said actuating module.

13. The injection device as claimed in claim 1, further comprising a flexible printed circuit board having a main board portion and a folded portion that is connected to and disposed over said main board portion, said main board portion and said folded portion both being configured to allow electronic components to be installed thereon.

14. An injection device adapted for adhering to the skin of a user to inject a liquid into the user, said injection device comprising:
an adhesive patch having a patch body and a hollow region that is formed in said patch body, said patch body having opposite first and second surfaces;
a catheter extending at an angle to said adhesive patch from said first surface, through said hollow region, to a side of said adhesive patch corresponding to said second surface; and
a release member removably covering said second surface of said patch body.

15. An injection device adapted for adhering to the skin of a user to inject a liquid into the user, said injection device comprising:
an adhesive patch having a patch body and a hollow region that is formed in said patch body, said patch body having opposite first and second surfaces; and
a catheter having a secured end portion that is disposed at said first surface of said patch body and an injection end portion that is disposed at said second surface of said patch body and for piercing the skin of the user.
